(19) United States
(12) Patent Application Publication
(10) Pub. No.: US 2010/0099775 A1
(43) Pub. Date: Apr. 22, 2010

(54) METHOD FOR AMELIORATING OF POST-ANESTHETIC RECOVERY

(75) Inventors: Joseph Schwarz, Markham (CA); Michael Weisspapir, Markham (CA)

Correspondence Address: ALPHARX INC., 168 KONRAD CRESCENT, SUITE 200, MARKHAM, L3R 9T9, CA

(73) Assignee: ALPHARX INC., Markham (CA)

(21) Appl. No.: 12/253,667

(22) Filed: Oct. 17, 2008

(51) Int. Cl. A61K 31/122; A61P 25/00

(52) U.S. Cl. 514/678

(57) ABSTRACT

The invention describes use of injectable form of Idebenone to improve recovery after general anesthesia and of cognitive functions, suppress disorientation and other signs of neuronal damage.

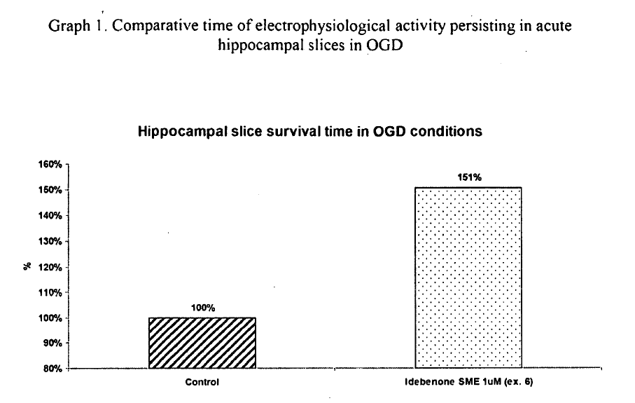

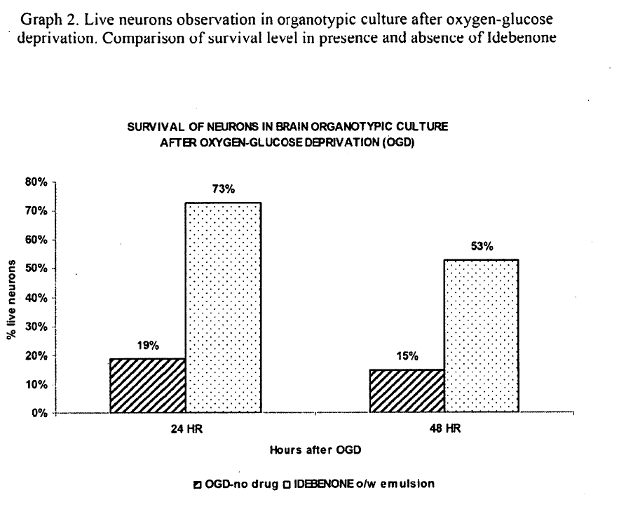

### FIELD OF INVENTION

The invention relates to the field of preparation of stable formulations of Idebenone, suitable for parenteral administration. Existing oral dosage forms of Idebenone associated with high metabolization in liver (“first pass effect”) can not be administered in acute situation or in case of patient unconsciousness. Development of injectable form of Idebenone is highly demanded.

### BACKGROUND OF THE INVENTION

Post-Operative Cognitive Dysfunction (POCD) syndrome is common, especially in seniors, undergoing extensive surgical procedures such as cardiac surgery or hip-replacement. There are over 2.5 million such surgical procedures annually in North America with an incidence of POCD of over 30%. There is serious demand for intervention and yet no adequate treatment options exist for this distressing post-surgical event. Post-Operative Cognitive Dysfunction (POCD) are significant and serious consequences of extensive surgical procedures in seniors, including cardiac surgery, hip replacement and many other serious surgical procedures. In cardiac surgery alone, there are over 2 million surgical procedures annually in the US. Post anesthetic recovery from long-acting anesthetics often leaves patients in a state of marked disorientation and impaired cognitive function for prolonged periods of time. Even the advent of newer short-acting anesthetic medicines does not alleviate the post-anesthetic effects on elderly surgical patients [1]

The incidence of serious post-operative adverse events, including cognitive dysfunction, delirium and stroke, reach as high as 30-35% of these surgical procedures, resulting in extensive prolonged hospital stays and serious quality of life issues for the patient and their healthcare providers. The ability to reduce post-anesthetic stroke from ˜2.5% to 1.5%, or to reduce post-operative cognitive dysfunctions substantially from ˜30% would mean significant inroads in both cost-savings and quality of life management. There is now substantial evidence that many elderly patients experience cognitive deterioration postoperatively. In a prospective, randomized trial of general vs epidural anesthesia with sedation for total knee replacement in patients >70 yr of age, cognitive performance, as assessed with psychometric tests, was worse than the preoperative baseline in 4-6% of patients six months after anesthesia and surgery. Another large, prospective, controlled international study demonstrated a cognitive dysfunction in 9.9% of patients three months postoperatively whereas only about 3% of the age-matched controls were similarly impaired. Among patients over 75 yr of age, 14% had a persistent cognitive dysfunction after general anesthesia and surgery. [2]

Antioxidants are offered to be used as protective agents to diminish brain damage caused by extended anesthesia. Various substances—antioxidants and radical scavengers—were tested in vitro in cell cultures, ex vivo in brain slices and in vivo in animal models. In such experiments Idebenone, 2,3-dimethoxy-5-methyl-6-(10-hydroxydecyl)-1,4 benzoquinone, demonstrated pronounced antioxidant activity and marked protection against oxidative damage of brain cells. Oral form of Idebenone is used for treatment of cardiac muscle athrophy in Friedreich's Ataxia, as a liver protectant and in some extent in treatment of Alzheimer's disease [U.S. Pat. No. 5,916,925 “Pharmaceutical composition for treatment of dementia”.]

In a small human study of nine patients with cerebrovascular disease, 90 mg idebenone was given daily, and electroencephalograms and clinical symptoms were monitored. The results suggested that Idebenone supplementation produced improvements in EEG and clinical symptoms in these patients [3].

Idebenone protects cultured cortical neurons against necrotic degeneration; it rescued cortical neurons even when applied 30 min after the NMDA pulse, suggesting that the drug interferes with the chain of toxic reactions triggered by an excessive stimulation of excitatory amino acid receptors [4]

Idebenone oral dosing (5 mg/kg daily for 8 weeks) in Friedreich's Ataxia patients significantly decreased a marker of oxidative DNA damage [5]. Idebenone prevented iron-induced lipoperoxidation and cardiac muscle injury in three patients given 5 mg/kg daily for 4-9 months, resulting in a reduction of left ventricular enlargement in these patients. [6]

In experiments in cell cultures idebenone can scavenge a variety of free radical species [7]. Idebenone can also redox couple with hypervalent species of myoglobin or hemoglobin, thus preventing lipid peroxidation promoted by these species. Likewise, idebenone inhibits microsomal lipid peroxidation induced by ADP-iron complexes or organic hydroperoxides. In so doing, idebenone prevents the destruction of cytochrome P450, which otherwise would accompany lipid peroxidation.

It has been reported that Idebenone ameliorates learning and memory disturbances in experimental models produced by cerebral embolization, cerebral ischemia or lesions in the basal forebrain in rats, which is the area of origin of the acetylcholine neuron system projecting into the cerebral cortex, hippocampus and amygdala. In clinical tests, idebenone was recognized to be effective in psychological dysfunctions such as a decline of memory retention and disorientation [8].

Bioavailability of oral Idebenone is relatively high due to polar hydrophobic nature of the molecule. However, oral administration of Idebenone accompanies with pronounced first pass metabolism of Idebenone in liver, and only small amount of the drug can reach the brain or other targeted organ. Additionally, effects of the oral treatment become apparent after several weeks or even months of the drug use.

Injectable form of Idebenone can overcome first pass effect of the oral dosage form and provides the required concentration in blood and brain fast. Nevertheless, there is no one example of Idebenone dosage form, suitable for parenteral delivery. The only described case of inravenous administration of Idebenone to rats [8] utilized 10% solution of polyethoxylated castor oil surfactant HCO-60, what is absolutely not applicable for human use due to extreme hemolytic properties of such vehicle.

Low water solubility of Idebenone makes this task very difficult. Use of water miscible solvents (alcohol, propylene glycol, liquid PEG, N-methylpyrrolidone, etc.) where Idebenone dissolves well is inappropriate due to immediate drug precipitation upon contact with physiological fluids or water phase. Inclusion complex of Idebenone with cyclodextrin is described, but it is water dispersible, not soluble, and not suitable for injection. Solubility of Idebenone in fixed oils (soy, corn, almond, etc.) is low; drug precipitates from such emulsion during storage limiting possibility of using them for preparation of emulsion based injectable form of the drug.

### BRIEF DESCRIPTION OF THE INVENTION

An objective of the present invention is to provide an adequate method for protection of brain from functional impairment caused by extended anesthesia, using injectable formulation of Idebenone. Surprisingly it was found that stable Idebenone formulation, suitable for parenteral administration, provides noticeable protection of the brain tissues in case of cellular damage, associated with POCD. Such formulation was prepared by using oil-in water emulsion, made of mixture of distinct oily components. Idebenone concentration in formulations varied from 0.1% to 2% by weight. The oil composition of the emulsion was compounded in a manner that all incorporated Idebenone was completely dissolved in the discontinuous (oil) phase of the emulsion, avoiding drug precipitation during storage and providing stable formulation.

Formulation were administrated via intravenous, intraperitoneal or subcutaneous injections during in vivo tests, or added after required dilution to cell culture media during “in vitro” or “ex vivo” experiments, and demonstrated excellent biocompatibility, absence of irritation or toxicity signs and pronounced brain tissues protection.

The following examples are intended to illustrate certain preferred embodiments of the invention and no limitation upon the invention is implied by their inclusion

### Idebenone Formulations

### Example 1-10

### Idebenone in Oil-in-Water Emulsions

### Example 1

### Preparation of Injectable Idebenone o/w Emulsion

Oil components of the formulation (Capric/caprylic triglycerides, acetylated monoglycerides and D-alpha-Tocopherol USP) were combined with lecithin and ethoxylated castor oil and mixed at 40° C. for 1 hour. Idebenone was dissolved in warm mixture of oils and surfactants and then blended with water phase, comprising water, EDTA and Glycerin using high shear rotor-stator mixer (5-10,000 rpm, 2 minutes). Obtained emulsion was treated with high pressure homogenizer (e.g., Avestin™ Emulsiflex C5) at 5,000-15,000 psi (300-1000 bar) for 3-5 cycles. After cooling to room temperature emulsion was filtered through sterile microporous membrane filter (0.2 mcm or 0.45 mcm) in aseptic conditions and dispensed into sterile glass vials. Sealed vials were stored in refrigerator or at room temperature, protected from light.

Idebenone content was tested using HPLC method.

Examples 2-10 of Idebenone loaded o/w emulsions were prepared in similar manner, excluding example 8 where instead of high pressure homogenization the mixture of the oil and water phases was passed through 0.22 mcm microporous membrane 3 times. Compositions of examples 1 through 10 are presented in table 1.

TABLE 1
Idebenone in oil-in-water emulsions (Examples 1-10)

| | 1 | 2 | 3 | 4 | 5 | 6 | 7 | 8 | 9 | 10 |
|---|---|---|---|---|---|---|---|---|---|---|
| | Percentage of composition | | | | | | | | | |
| Idebenone | 1.0 | 2.0 | 1.0 | 0.25 | 0.10 | 0.35 | 2.0 | 0.5 | 0.5 | 2.0 |
| Soya oil | | | 5.0 | 2.0 | | | | | | |
| Capric/caprylic | 12.5 | 18.0 | 10.0 | 8.0 | 10.0 | | 12.0 | | 16.0 | 18.0 |
| triglycerides (MCT) | | | | | | | | | | |
| Tocopherol USP | 0.1 | 8.0 | | | | | 3.0 | 2.5 | | 2.0 |
| Acetylated monoglycerides | 12.5 | | 10.0 | | | 10.0 | 15.0 | | 9.0 | 10.0 |
| Ethyl oleate | | | | | | | | 5.0 | | |
| Polysorbate-80 | | 1.0 | | | | | 0.5 | | 0.1 | 0.5 |
| TPGS | | | 0.5 | | | | | 1.0 | | |
| Ethoxylated castor oil | 0.5 | | | | 0.25 | | | | | |
| Lecithin USP | 2.0 | 1.5 | 1.8 | 1.0 | 1.0 | 2.0 | 1.5 | 1.0 | 1.5 | 1.5 |
| (phosphatidylcholine > | | | | | | | | | | |
| 70%) | | | | | | | | | | |
| Propylene glycol | | | | | | | | | | |
| Glycerin | 2.5 | 2.25 | | 2.25 | 2.25 | | | | 2.25 | 2.25 |
| Glycine | 0.5 | | | | 0.5 | | | 2.0 | | |
| EDTA | 0.02 | 0.02 | 0.02 | 0.02 | 0.02 | 0.02 | 0.02 | 0.02 | 0.02 | 0.02 |
| PURIFIED WATER | 68.38 | 67.23 | 69.88 | 86.48 | 85.88 | 87.63 | 64.48 | 87.98 | 70.63 | 63.73 |
| to 100% | | | | | | | | | | |
| Total: | 100% | 100% | 100% | 100% | 100% | 100% | 100% | 100% | 100% | 100% |

### Animal Experimental Protocol

Animals (Fisher 344 rats) were trained to perform memory testing tasks using a 12-limb radial maze device 24 hours before the start of the experimental procedures. Animals were divided into two groups: a control group that was injected with vehicle and experimental group, treated with injectable Idebenone. All animals were anesthetized to a certain level with the inhalational anesthetic isoflurane for one hour without any surgical intervention. The cognitive behavioral testing and memory testing designed to determine the post anesthesia change in locomotor activity and choice accuracy in a working memory tasks at 4, 24 and 48 hours after.

Animals were given Idebenone intraperitoneally in dose of 10 mg/kg 15 minutes before anesthesia. Observations confirmed significant improvement of cognitive functions and working memory in group given Idebenone injection compared with control group for all observed time points. Awakening time after general anesthesia substantially decreases for animals given Idebenone injection.

### Oxygen-Glucose Deprivation in Acute and Organotypic Hyppocampal Slices

The neuroprotective effects of Idebenone were evaluated in hippocampal slice cultures, which enable relatively long-term assessment of the survival of several different neuronal populations. Hippocampal slices were prepared from 14-20 day old Wistar rats after the entire hippocampus was removed and slices (400 μm) were made with a Mac-Ilwain tissue chopper. Cell viability was assessed by control of electrophysiological activity. Organotypic slices (250-300 mcm thickness) were grown using standard methods; then transferred onto 30-mm diameter membrane inserts and put into 6-well culture trays with 1.5 mL of slice culture medium per well. Slices were kept in culture for 7-14 days before study. In vitro ischemia was simulated by hypoxia combined with glucose-free media (Oxygen-Glucose Deprivation, OGD). Before hypoxia, the slices were washed three times with glucose-free Hank's balanced salt solution (HBSS). The cultures were then placed into an airtight Incubator chamber through which 95% N2/5% CO2 gas preheated to 37° C. was passed at 5-10 L per minute. The temperature of the chamber was kept at 37° C., and the partial pressure of oxygen was approximately 0-0.2 mm Hg. For studies involving Idebenone formulations, slices were rinsed with glucose-free HBSS containing Idebenone in submicron emulsion, diluted to obtain 0.1 or 1.0 μM concentrations in culture; the drug remained in contact with the cultures for the duration of the OGD. After the insult, the culture tray was removed from the chamber, the anoxic-glucose-free HBSS was aspirated from the wells, and standard (oxygenated) slice culture media was added. Cell viability was assessed by propidium iodide staining.

Use of Idebenone in submicron emulsion (SME) increased survival time in acute hippocampal slices for more than 50%, from 7.0 to 10.6 minutes (see Graph 1):

| Test article | Electrophysiological activity in OGD |
|---|---|
| Control vehicle (diluted o/w | 7.0 minutes |
| emulsion) | |
| Idebenone 1 micromol/L in o/w | 10.6 minutes |
| emulsion (example 6) | |

Administration of injectable form of Idebenone increased survival of neurons in brain organotypic culture slices after OGD several times (see Graph 2).

1. Method of treatment or prevention of post-operative cognitive dysfunction (POCD) syndrome or delirium and improvement in post-anesthesia recovery by the parenteral administration of a pharmaceutical composition, comprising at least one physiologically acceptable ubiquinone.

2-6. (canceled)

7. A method as set forth in claim 1 wherein said ubiquinone is 2,3-dimethoxy-5-methyl-6-(10-hydroxydecyl)-1,4-benzoquinone (Idebenone).

8. A method as set forth in claim 1 wherein said composition is administrated parenterally via intravenous injection, intravenous infusion, intra-arterial, intramuscular, subcutaneous or intra-peritoneal injection.

9. A method as set forth in claim 7 wherein said Idebenone is administrated in doses from 0.5 to 50 mg/kg per day.

10. A method as set forth in claim 1 wherein said composition is a colloidal delivery system, selected from micellar preparations, emulsions, solid lipid nanoparticles or suspensions, wherein said ubiquinone is associated with the hydrophobic phase of the colloidal system.

11. A method as set forth in claim 10 wherein said colloidal delivery system is an oil-in-water emulsion.
